(19) United States
(12) Patent Application Publication
(10) Pub. No.: US 2018/0256925 A1
(43) Pub. Date: Sep. 13, 2018

(54) METHOD AND APPARATUS FOR PROVIDING POWER FOR A DEVICE

(71) Applicant: Illinois Tool Works Inc., Glenview, IL (US)

(72) Inventor: Eric Sommers, Appleton, WI (US)

(21) Appl. No.: 15/455,675

(22) Filed: Mar. 10, 2017

(51) Int. Cl. A62B 18/04; A61F 9/06; A62B 7/12; A62B 9/00; A62B 18/00; A42B 3/04; A42B 3/22

(52) U.S. Cl. CPC A62B 18/04; A61F 9/068; A62B 7/12; A42B 3/044; A62B 18/006; A42B 3/0406; A42B 3/225; A62B 9/006

(57) ABSTRACT

Provided is a disclosure for a head protection device that includes an air providing unit configured to provide air to a user, an air conduit configured to transport the air from the air providing unit to the head protection device, a power connector configured to receive power via electrical wiring from a power source that is not attached to the head protection device.

### BACKGROUND

The present disclosure relates to providing power, and more particularly, to a method and apparatus for providing power for work equipment.

Some environments, such as, for example, a welding environment, a cave or other enclosed areas, etc., can sometimes be dark and/or have contaminated air, and/or the environment may also be warm. Accordingly, in such cases cooling air, air filtration, and/or lighting may be needed for an extended period of time.

Limitations and disadvantages of conventional systems for a work environment will become apparent to one of skill in the art through comparison of such approaches with some aspects of the present method and apparatus set forth in the remainder of this disclosure with reference to the drawings.

### SUMMARY

Methods and apparatus are provided for providing power for work equipment, substantially as illustrated by and described in connection with at least one of the figures, as set forth more completely in the claims.

### BRIEF DESCRIPTION OF THE DRAWINGS

These and/or other aspects will become apparent and more readily appreciated from the following description of some example embodiments, taken in conjunction with the accompanying drawings.

FIG. 1A is an example head protection device with a light, and an air source, in accordance with an embodiment of the disclosure.

FIG. 1B is an example head protection device where a power cable is integrated with an air tube, in accordance with an embodiment of the disclosure.

FIG. 1C is an example connection for auxiliary power using an integrated power cable, in accordance with an embodiment of the disclosure.

FIG. 2A is an example of an air providing unit of FIG. 1A plugged into a wall, in accordance with an embodiment of the disclosure.

FIG. 2B is an example of an air providing unit of FIG. 1A being charged wirelessly, in accordance with an embodiment of the disclosure.

FIG. 3 is an example configuration of a device with a separate battery, in accordance with an embodiment of the disclosure.

FIG. 4 is a block diagram of an air providing unit communicating with a head protective device, in accordance with an embodiment of the disclosure.

The figures are not necessarily to scale. Where appropriate, the same or similar reference numerals are used to refer to the same or similar components.

### DETAILED DESCRIPTION

Various embodiments of the disclosure are described that provide power for various equipment that a user/worker, such as, for example, a welder may use.

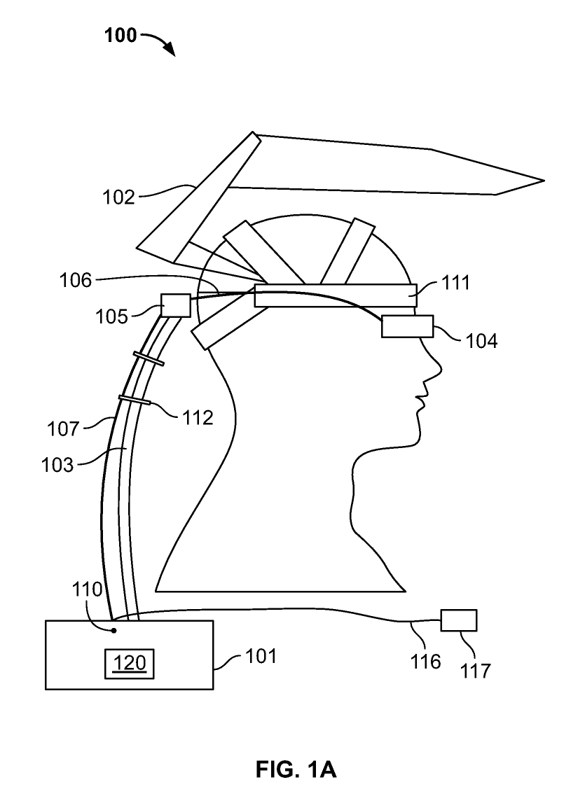

FIG. 1A is an example head protection device with a light, and an air source, in accordance with an embodiment of the disclosure. Referring to FIG. 1, there is shown the head protection device 100, which, as an example, is shown a welding helmet worn by a user. The environment in which the head protection device 100 may be used is a gaseous environment for welding, grinding, cutting, etc. These types of work may generally be referred to as work done in a welding environment, and may generate noxious fumes and/or particulates around the user.

The head protection device 100 comprises a shield assembly 102 that is attached to a headgear 111 at the attachment point 111a. The headgear 111 is worn on the head and the shield assembly 102 can, for example, rotate about the attachment point 111a to raise the shield assembly 102 or lower the shield assembly 102.

Air can be provided to the head protection device 100 via an air conduit 103. The air is provided by, for example, an air providing unit 101. The air providing unit 101 may be, for example, a PAPR (Powered Air Purifying Respirator) or a fan. Accordingly, there may be positive air pressure in the head protection unit 100 with respect to the environment that the user/worker is in.

This may alleviate seeping in of environmental air into the head protection device 100. The conduit 103 may attach to a vent (not shown), where the vent may provide/direct the air to appropriate portions of the head protection device 100. A battery 120, which may be rechargeable, may provide power for the air providing unit 101. The battery 120 may also be able to provide power to an electrical device (such as, for example, the air providing unit 101 or an auxiliary device 104) while electrically connected to a charging unit.

The head protection device 100 may also have an attached auxiliary device 104. The auxiliary device 104 may be, for example, a light that enables the user/worker to see his work area. The auxiliary device 104 may also be other devices such as, for example, heads up displays (HUDs) within helmet, communication equipment (for example, to talk to others, to adjust machine settings), augmented reality devices, etc. A HUD may be used by, for example, a welder to see various parameters for a welding machine, welding speed, environmental conditions (of the surrounding air such as temperature, humidity, type of contaminants, amount of particulates in the air, and the like), etc. Augmented reality may also be projected onto a HUD when the environment may be hazy, for example, due to smoke from welding. A HUD may also be used by, for example, a motorcyclist, as well as augmented reality for the motorcyclist. Communication equipment may be used for interpersonal communication as well as to allow control of various devices being used.

For example, the environment may be monitored and commands sent to the air providing unit 101 to vary air filtration and/or air speed. Commands may also be sent to control light output, where the light output and/or direction may depend on various environmental parameters such as, for example, brightness, the amount of smoke in the environment, etc.

The auxiliary device 104 can receive power via an auxiliary device power cable 106, which may be electrically connected to the power junction box 105. The power junction box 105 may have at least one power connector to which various devices can be plugged in, and at least one power connector may be a universal serial bus (USB) connector. The power junction box 105 is electrically connected in turn to the air providing unit 101 via the power cable 107. The power junction box 105 may be able to provide one or more DC voltages via the power connectors. Accordingly, the power junction box 105 may receive the various voltages from the air providing unit 101, or the power junction box 105 may have appropriate conversion circuitry for DC-DC voltage conversion.

The power cable 107 can be electrically connected to the air providing unit 101 via a power receptacle 110. The power cable 107 may plug into the power receptacle 110 or be hardwired to the power receptacle 110. Additionally, the power receptacle 110 at the air providing unit 101 may also provide power via another power cable 116 that has at least one power connector 117. The power connector 117 may also be similar to the power junction box 105.

In some embodiments, the power cable 107 may be fastened to the air conduit 103 via a fastening device 112, such as, for example, a cable tie. In other embodiments, the power cable 107 may be a part of the air conduit 103. Accordingly, the power cable 107 may not need to be fastened to the air conduit 103 by the fastening device 112 since it is integrated with the air conduit 103.

Also, while the head protection device 100 has been described as being attached to the air providing unit 101, various embodiments need not be so limited. For example, the head protection device 100 may be attached to another type of work device or tool.

Various embodiments of the disclosure may allow the power source for one or more auxiliary devices 104 mounted on the head protection device 100 to be placed elsewhere other than the head protection device 100. This allows reduction of weight carried by the head protection device 100, thereby easing strain on the neck muscles of a user/worker to allow a more comfortable use/work condition. Additionally, the auxiliary device can operate for a longer time due to the external power source.

In this example embodiment, the head protection device 100 was shown to be a welding helmet. However, in other embodiments, the head protection device 100 may be used in a welding type environment such as, for example, for grinding, cutting, etc. Also, various embodiments of the disclosure may also be used, for example, as a motorcycle helmet, fireman's helmet, riot helmet, etc. In various embodiments, the shield of the head protection device may not be completely rigid as in a welding helmet or a motorcycle helmet. In some embodiments, the shield may be a full-face shield while in other embodiments the shield may cover a part of the face. In some embodiments, the shield may be fixed in place and not rotatable up and down. As an example, the head protection device 100 may be a part of an isolation suit worn to keep out contaminants, hazards, etc.

FIG. 1B is an example head protection device where a power cable is integrated with an air tube, in accordance with an embodiment of the disclosure. Referring to FIG. 1B, there is shown the auxiliary device 104 receiving power via the auxiliary device power cable 106. The auxiliary device power cable 106 is electrically connected to the power junction box 105, which is electrically connected to the integrated power cable 108a. The integrated power cable 108a is a part of the air conduit 103. The integrated power cable 108a may electrically connect to the air providing unit 101 for power.

The air conduit 103 may attach to the air providing unit 101 via the air conduit connection 114. Accordingly, the integrated power cable 108a may also make an electrical connection with the air providing unit 101 when the air conduit 103 is attached to the air providing unit 101, or the integrated power cable 108a may have a connector that can be used to electrically connect to the air providing unit 101 or directly to a battery for the air providing unit 101.

Additionally, various embodiments may provide the air providing unit 101 and/or the battery 120 with multiple connectors for providing power. Accordingly, an embodiment may allow a device to draw power directly from the battery 120 by plugging into a connector in the battery 120, or draw power from the battery 120 by plugging into a connector in the air providing unit 101.

FIG. 1C is an example connection for auxiliary power using an integrated power cable, in accordance with an embodiment of the disclosure. Referring to FIG. 1C, there is shown the air providing unit 101 and the air conduit 103. The air conduit 103 has, at the first end that attaches to the air providing unit 101, a pair of electrical contacts 108b. The air providing unit 101 has at its receptacle for receiving the air conduit 103 a pair of electrical contacts 115. Accordingly, when the air conduit 103 is mated to the air providing unit 101, the electrical contacts 108b and 115 make electrical connection to be able to provide electric power via the integrated power cable 108a. Various embodiments may also allow for an electrical connector (not shown) on the integrated power cable 108a that can be electrically connected directly to a battery or power source for the air providing unit 101. A second end of the air conduit 103 may mate to the head protective unit 100.

For ease of explanation, the integrated power cable 108a is shown as being placed outside the air conduit 103. However, in various embodiments, the integrated power cable 108a may be integrated with the air conduit 103 or held in place by, for example, snaps (not shown) that are part of the air conduit 103. When snaps are in place in the air conduit 103, the integrated power cable 108a may be held in place by the snaps, where the integrated power cable 108a may be entirely or in part encased by the snaps. In some embodiments, the integrated power cable 108a may be encased, for example, by plastic that may be used to form the air conduit 103. Accordingly, the integrated power cable 108a may be on an external surface of the air conduit 103, on an internal surface of the air conduit 103, and/or embedded in the material of the air conduit 103.

When the air conduit 103 is attached to the head protection device 100, there may be a similar electrical connection as with the electrical contacts 108b and 115, or there may be a separate electrical connection on the head protection device 100.

Various embodiments may have the air conduit 103 detachably attached to the head protection device 100 and/or the air providing unit 101. Accordingly, when the air conduit 103 is attached to the head protection device and the air providing unit 101, electric power may be available via the integrated power cable 108a.

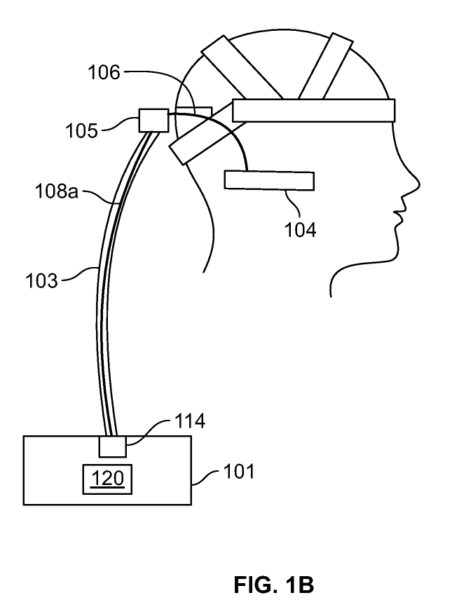

FIG. 2A is an example of an air providing unit of FIG. 1A plugged into a wall, in accordance with an embodiment of the disclosure. Referring to FIG. 2A, there is shown a device, such as, for example, the air providing unit 101 that is plugged into a wall socket 202 in the wall 200. The air providing unit 101 may have an electric cord 210 with an electric plug 212, where the electric plug 212 plugs into the wall socket 202. The air providing unit 101 may be plugged in, for example, to recharge a battery 220 that provides power for the air providing unit 101. The battery 220 may also be able to provide power to the air providing unit 101 and/or the auxiliary device 104 while the electric plug 212 is plugged into the wall socket 202, whether the battery 220 is fully charged or not.

FIG. 2B is an example of an air providing unit of FIG. 1A being charged wirelessly, in accordance with an embodiment of the disclosure. In FIG. 2B, there is shown the battery 220 being charged wirelessly by the wireless charger 230. The wireless charger 230 may be plugged to the wall socket 202 via the electric plug 212.

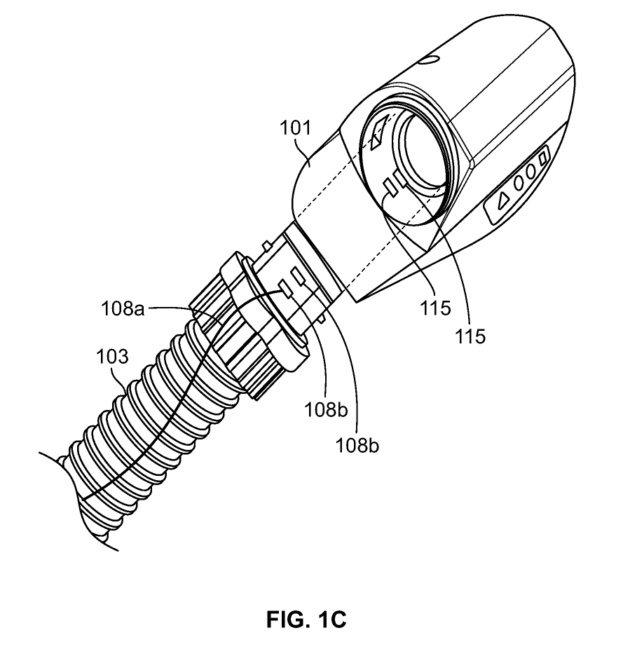

FIG. 3 is an example configuration of a device with a separate battery, in accordance with an embodiment of the disclosure. For example, the air providing unit 101 may have a separate battery 302, where the battery 302 may be similar to the battery 220. Accordingly, the battery 220 may be charged via wire or wirelessly similarly as described with respect to FIGS. 2A and 2B. Additionally, the battery 302 may also transmit power to the air providing unit 101 via wire or wirelessly (as shown by the dashed line). The battery 302 may also be electrically connected to the air providing unit 101 such that there is an electrical connection.

Accordingly, the battery 302 may be carried by the user/worker near the air providing unit 101 such as on a belt that carries the air providing unit 101 and the battery 302. The battery 302 may also be carried farther away on the body of the user/worker from the air providing unit 101, or not carried by the user/worker. When the battery 302 is not carried by the user/worker, the battery 302 may be, for example, placed in a nearby location such as a work table (not shown).

Whether a battery is part of the air providing unit 101 (battery 220) or an external battery (battery 302), the power cable 107 or the integrated power cable 108a may plug directly into the battery 302/107 or to the air providing unit 101.

Various embodiments of the disclosure may have as separate units the battery 302, the air conduit 103, and the auxiliary devices 104 on a head protection device 100, where power for the auxiliary devices 104 is provided via an electrical cable that is plugged into the battery 302 or to the air providing unit 101. The air providing unit 101 may be plugged into the battery 302 for power.

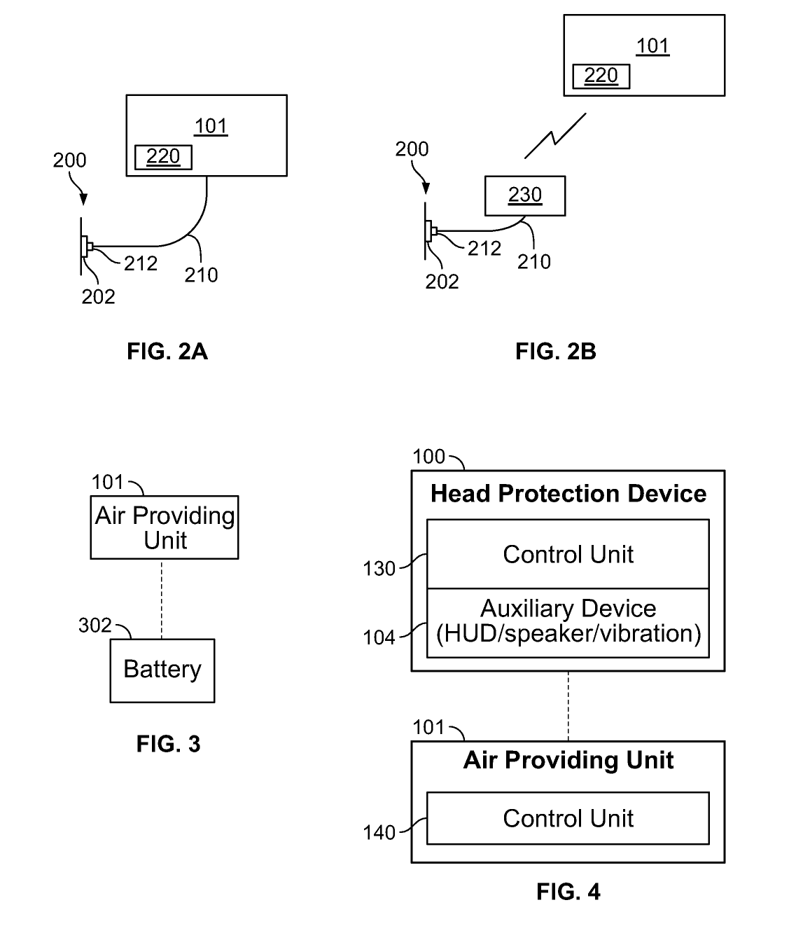

FIG. 4 is a block diagram of an air providing unit communicating with a head protective device, in accordance with an embodiment of the disclosure. Referring to FIG. 4, there is shown the air providing unit 101 configured to communicate with the head protection device 100. The communication may be via, for example, the control units 130 and 140. The control units 130 and 140 may comprise circuitry to enable transmission and reception of wireless and/or wired signals using an appropriate protocol such as, for example, USB protocol, cellular protocol, Wi-Fi protocol, Bluetooth protocol, etc. Wired communication may be provided, for example, via the power cable 107 or a separate communication cable.

The control units 130 and 140 may comprise transceiver block, processing block (for example, a processor, controller, etc.), memory block (including, for example, volatile memory and non-volatile memory), as well as various interfaces such as, for example, a USB connector and/or antenna(s).

The control unit 130 may, for example, monitor environmental conditions such as air quality, air temperature, humidity, brightness, etc., and transmit the monitored information to the control unit 140. The control unit 140 may then determine changes to the air providing unit 101. For example, changes to air filtering may be provided if different levels of filtering are available, or the air speed may be changed. The control unit 140 may also determine whether the auxiliary device 104 (which may be a light) may need to have light intensity changed or the angle of the light changed.

Additionally, if there is an auxiliary device 104 that may have HUD or augmented reality capabilities, the control unit 140 may provide heads-up information or augmented reality information to be displayed on the auxiliary device 104. The HUD output may be displayed, for example, on a visor portion of the shield assembly 102. Other control signals may be sent by the control unit 140 that may provide audible outputs and/or vibration outputs by the head protection device 100. A vibrating unit and/or a speaker may be built in as a part of the head protection device 100 or may be a part of an auxiliary device 104.

While a specific configuration was described, various embodiments of the disclosure may have different functionality performed by the control units 130 and 140, and/or by other control units not described here. For example, the control unit 140 may be a part of another device such as, for example, the battery 120, or another external unit that communicates wirelessly (or via wire) with other control units such as, for example, the control unit 130.

An embodiment of the disclosure is a head worn device that includes an air conduit configured to provide air from a powered air providing unit to the head worn device at a positive air pressure. The head worn device may have an electrical connector configured to receive electric power from a power source of the powered air providing unit via an electrical circuit. In an embodiment of the disclosure, the power source is not attached to the head worn device. The head worn device may be used for a variety of uses including, for example, welding, cutting, and grinding.

The electric connector may be detachably attached to the head worn device to allow removal or attachment of the electrical connector. In some embodiments, the electrical connector may be more firmly attached, permanently in some embodiments, to the head worn device.

The electrical circuit may be attached to the air conduit either detachably or as an integral part of the air conduit. When the air conduit is attached to the head worn device and the powered air providing unit, power may be provided to one or more electrical outlets at the electrical connector. At least one electrical outlet is a USB outlet capable of accepting a USB connector. In some embodiments, the electrical circuit may electrically connect directly to the power source.

An auxiliary device, such as a light, for example, may be electrically connected directly to the electrical circuit or directly to the power source.

The powered air providing unit may be, for example, a powered air purifying respirator (PAPR) or a fan, and the power source can be a battery. The battery may be rechargeable and also may be able to supply power to one or more electrical devices (for example, to the powered air providing unit and/or the PAPR) while it is electrically connected to a charging unit.

The head worn device may comprise a first transceiver configured to communicate information via the electrical wiring to a second transceiver. The second transceiver may communicate information to/from the first transceiver such that the first transceiver can output the received information as one or more of a visual output, an audio output, and a vibratory output. Some embodiments of the disclosure may have a face shield for the head worn device. The face shield may be of various types from flexible to very rigid, from providing light duty shielding to heavy-duty shielding, from partially shielding the face to completely shielding the face, etc. The visual output may be displayed, for example, on a visor of the head worn device as a heads-up display.

Another embodiment of the disclosure may be a head worn device with a face shield, an air conduit configured to provide air from a powered air providing unit to the head worn device at a positive air pressure, a power source configured to provide power to the powered air providing unit via a first electrical circuit, where the power source is not mounted to the head worn device, and a second electrical circuit configured to provide power from the power source to an auxiliary device. The system may comprise an electrical connector electrically connected to the second electrical circuit, wherein the auxiliary device receives power via the electrical connector.

Still another embodiment of the disclosure may be a power source comprising a first electrical circuit configured to provide power from the power source to the air providing device and a second electrical circuit configured to provide power from the power source to an auxiliary device. At least one of the first electrical circuit and the second electrical circuit may electrically connect to the power source via a socket at the power source. And at least one of the first electrical circuit and the second electrical circuit may be attached to an air conduit that is configured to provide air from the air providing device to a head worn device. The power source may not be attached to the head worn device, and the auxiliary device may be a light source that is detachably attached, for example, to the head worn device.

Various embodiments have disclosed providing power to various equipment. However the present disclosure is not limited to any specific type of equipment. For example, a head protection device may be a motorcycle helmet that may have a fan/PAPR and/or an auxiliary device such as a light attached to it. The light may be used while riding a motorcycle, or when the motorcyclist gets off the motorcycle. For example, if the motorcyclist needs to work on his motorcycle, or if he is looking for something, the helmet attached light may be useful. Other types of auxiliary device may be, for example, DC-power hand tools.

As utilized herein, “and/or” means any one or more of the items in the list joined by “and/or.” As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”. As utilized herein, the term “exemplary” means serving as a non-limiting example, instance, or illustration. As utilized herein, the terms “e.g.” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled or not enabled (e.g., by a user-configurable setting, factory trim, etc.).

While the present method and/or system has been described with reference to certain implementations, it will be understood by those skilled in the art that various changes may be made and equivalents may be substituted without departing from the scope of the present method and/or system. In addition, many modifications may be made to adapt a particular situation or material to the teachings of the present disclosure without departing from its scope. Therefore, the present method and/or system are not limited to the particular implementations disclosed. Instead, the present method and/or system will include all implementations falling within the scope of the appended claims, both literally and under the doctrine of equivalents.

What is claimed:

1. A head worn device comprising:
an air conduit configured to provide air from a powered air providing unit to the head worn device at a positive air pressure; and
an electrical connector on the head worn device configured to receive electric power from a power source of the powered air providing unit via an electrical circuit,
wherein the power source is separate from the head worn device.

2. The head worn device of claim 1, wherein the head worn device is at least one of a welding helmet, a cutting helmet, and a grinding helmet.

3. The head word device of claim 1, wherein the electrical connector is detachably attached to the head worn device.

4. The head worn device of claim 1, wherein the electrical circuit is attached to the air conduit.

5. The head worn device of claim 4, wherein the air conduit is detachably attached to at least one of the head worn device and the air providing unit.

6. The head worn device of claim 5, wherein when the air conduit is detachably attached to the head worn device and the air providing unit, the electric power is available to at least one electrical outlet electrically connected to the electrical connector.

7. The head worn device of claim 1, wherein the electrical connector comprises electrical outlets, and the electrical outlets are configured to provide electric power when electrically connected to the power source.

8. The head worn device of claim 7, wherein at least one of the electrical outlets is a universal serial bus (USB) outlet.

7. The head worn device of claim 1, wherein the electrical circuit electrically connects directly to the power source.

8. The head worn device of claim 1, wherein an auxiliary device electrically connects directly to one of the electrical circuit and the power source.

9. The head worn device of claim 8, wherein the auxiliary device is a light source.

10. The head worn device of claim 1, wherein the powered air providing unit is one of a powered air purifying respirator and a fan.

11. The head worn device of claim 1, wherein the power source is a battery.

12. The head worn device of claim 11, wherein the battery is rechargeable.

13. The head worn device of claim 11, wherein the battery is configured to be electrically connected to a charging unit while the battery is supplying electric power to one or more electrical devices.

14. The head worn device of claim 1, comprising a first transceiver configured to communicate information via the electrical circuit to a second transceiver.

15. The head worn device of claim 14, wherein the second transceiver receives the information from the first transceiver and outputs the information as one or more of a visual output, an audio output, and a vibratory output.

16. The head worn device of claim 15, wherein the visual output is displayed on a visor of the head worn device as a heads-up display.

17. The head worn device of claim 1, comprising a face shield configured to shield a face of a user of the head worn device.

18. A power source for an air providing device, comprising:
a first electrical circuit configured to provide power from the power source to the air providing device; and
a second electrical circuit configured to provide power from the power source to an auxiliary device.

19. The power source of claim 18, wherein:
at least one of the first electrical circuit and the second electrical circuit electrically connects to the power source via a socket at the power source;
at least one of the first electrical circuit and the second electrical circuit is attached to an air conduit that is configured to provide air from the air providing device to a head worn device;
the power source is separate from the head worn device; and
the auxiliary device is a detachably attached light source.

20. The power source of claim 18, wherein at least one of the first electrical circuit and the second electrical circuit is hardwired directly to the power source.

21. A system, comprising:
a head worn device with a face shield;
an air conduit configured to provide air from a powered air providing unit to the head worn device at a positive air pressure;
a power source configured to provide power to the powered air providing unit via a first electrical circuit, wherein the power source is not mounted to the head worn device; and
a second electrical circuit configured to provide power from the power source to an auxiliary device.

22. The system of claim 21, comprising an electrical connector electrically connected to the second electrical circuit, wherein the auxiliary device receives power via the electrical connector.
